(19) United States
(12) Patent Application Publication
(10) Pub. No.: US 2026/0337137 A1
(43) Pub. Date: Oct. 29, 2026

(54) SMALL MODULAR REACTOR DRIVEN LOW-TEMPERATURE METHANOL AND SUBSEQUENT CHEMICAL PRODUCTION PATHWAYS

(71) Applicant: NuScale Power, LLC, Corvallis, OR (US)

(72) Inventors: Francis Y. Tsang, Bellingham, WA (US); José N. Reyes, Jr., Corvallis, OR (US); Luis DePavia, Corvallis, OR (US)

(73) Assignee: NuScale Power, LLC, Corvallis, OR (US)

(21) Appl. No.: 19/242,473

(22) Filed: Jun. 18, 2025

Related U.S. Application Data
(60) Provisional application No. 63/661,074, filed on Jun. 18, 2024.

(51) Int. Cl. C07C 29/152; B01D 53/047; B01D 53/26; B01J 19/24; C01B 3/34

(52) U.S. Cl. CPC C07C 29/152; B01D 53/047; B01D 53/265; B01J 19/245; C01B 3/34; B01D 2257/108; B01D 2257/80; C01B 2203/0233; C01B 2203/0475; C01B 2203/048; C01B 2203/0495; C01B 2203/062; C01B 2203/1241

(57) ABSTRACT

An integrated energy system (IES) including a power plant is discussed herein. In some examples, the IES may include a power plant configured to generate steam, a Low-Temperature Methane Steam Reforming plant configured to receive at least a portion of the steam from the power plant to react with Methane within the Low-Temperature Methane Steam Reforming plant to produce Hydrogen, first Carbon Monoxide, and Carbon Dioxide, a first separation unit configured to separate the Hydrogen, the first Carbon Monoxide, and the Carbon Dioxide, a Solid Oxide Electrolysis Stack configured to receive at least a portion of the Carbon Dioxide and to produce second Carbon Monoxide and Oxygen, a second separation unit configured to separate the Carbon Dioxide from the second Carbon Monoxide, and a methanol synthesis reactor configured to receive at least a portion of the Hydrogen and at least a portion of the second Carbon Monoxide to produce Methanol.

### CROSS-REFERENCE TO RELATED APPLICATIONS

This application claims the benefit of U.S. Provisional Patent Application No. 63/661,074, filed Jun. 18, 2024, entitled “SMALL MODULAR REACTOR DRIVEN LOW-TEMPERATURE METHANOL AND SUBSEQUENT CHEMICAL PRODUCTION PATHWAYS” which are incorporated herein by reference in their entirety.

### TECHNICAL FIELD

The present technology is directed to nuclear reactor integrated energy systems (IESs) for energy production and green industrial applications, such as to IESs including one or more nuclear reactors (e.g., small modular nuclear reactors (SMRs)) coupled to a low-temperature methane steam reforming process for producing hydrogen gas and carbon dioxide gas for methanol production, and associated devices and methods.

### BACKGROUND

The energy production landscape has evolved rapidly in recent years, with a growing emphasis on decarbonization, sustainability, and resilience, driving the adoption of cleaner and more efficient forms of power production. While fossil fuels continue to play a significant role in global energy supply, there is a need for increased deployment of renewable energy, coupled with advancements in energy storage, grid modernization, and energy efficiency measures, to address the challenges of climate change and energy transition.

Cumulative carbon dioxide emissions are the dominant driver of climate change. The largest carbon emission sources are attributed to the hydrocarbon fuel combustion in electricity, heat generation, and chemical industry which contributes to nearly two-thirds of the total worldwide CO₂ emissions. The seven largest CO₂ emission industries in the world are: (1) power plants (coal, natural gas, oil fired), (2) oil refinery plants, (3) ammonia production plants, (4) chemical manufacturing and production plants, (5) cement production plants, (6) steel manufacturing plants, and (7) transportation. The International Energy Agency (IEA) reported that the CO₂ emission from industrial processes and energy combustion grew by 321 Mt in 2022.

Most methanol is currently produced from natural gas, where natural gas is used both as a feedstock and as a process fuel. The CO₂ emissions associated with producing one metric ton of methanol vary depending on the production method, but generally range from 0.5 to 1.4 tons of CO₂ equivalent per ton of methanol. Traditional methanol production, particularly using methanol derived from coal, can have a higher carbon footprint, while newer technologies like e-methanol, which uses renewable energy and CO₂, can significantly reduce emission. The World Economic Forum states that traditional methanol production from coal can emit up to 3 tons of CO₂ per ton of methanol while steam reforming (SR), a common method for methanol production, can emit between 0.5 and 1.4 tons of CO₂ equivalent per ton of methanol. In essence, the carbon footprint of methanol production is highly dependent on the feedstock and production method used, and traditional fossil fuel-based methods can have substantial emissions.

An energy system incorporates various energy conversion technologies, such as power plants, cogeneration (combined heat and power) systems, and distributed generation units (such as solar panels and wind turbines). These technologies convert primary energy sources into usable forms of energy, such as electricity, heat, steam, and mechanical power that can be used as secondary energy sources. Energy systems leverage a diverse range of energy resources, including renewable energy sources (such as solar, wind, and hydroelectric power), conventional fuels (such as natural gas and coal), and emerging technologies (such as hydrogen and biofuels). By combining multiple energy sources, these systems can enhance energy security and resilience.

### BRIEF DESCRIPTION OF THE DRAWINGS

The Detailed Description is set forth with reference to the accompanying figures. In the figures, the left-most digit(s) of a reference number identifies the figure in which the reference number first appears. The use of the same reference numbers in different figures may indicate similar or identical items. Furthermore, the drawings may be considered as providing an approximate depiction of the relative sizes of the individual components within individual figures. However, the drawings are not to scale, and the relative sizes of the individual components, both within individual figures and between the different figures, may vary from what is depicted. In particular, some of the figures may depict components as a certain size or shape, while other figures may depict the same components on a larger scale or differently shaped for the sake of clarity.

FIG. 1 illustrates the thermodynamics of Methane Steam Reforming reactions as functions of reformer temperature.

FIG. 2 is a schematic diagram of an integrated energy system for producing Methanol that includes a power plant system in accordance with at least some embodiments of the present disclosure.

FIG. 3 is a schematic diagram of an integrated energy system for producing Methanol that includes a power plant system in accordance with embodiments of the present disclosure.

FIG. 4 is a block diagram illustrating an exemplary Low-Temperature Methane Steam Reforming Plant for producing Hydrogen and Carbon Dioxide in accordance with embodiments of the present disclosure.

FIG. 5 is a block diagram illustrating the utilization of a Solid Oxide Electrolysis Stack, in accordance with embodiments of the present disclosure.

FIG. 6 depicts a block diagram illustrating an exemplary Methanol Production Plant for producing Methanol in accordance with embodiments of the present disclosure.

FIG. 7 is a schematic view of a nuclear power plant system including multiple nuclear reactors in accordance with embodiments of the present technology.

FIG. 8 is a partially schematic, partially cross-sectional view of a nuclear reactor system configured in accordance with embodiments of the present technology.

FIG. 9 is a partially schematic, partially cross-sectional view of a nuclear reactor system configured in accordance with embodiments of the present technology.

FIG. 10 illustrates an exemplary Low-Temperature Methane steam reforming process to produce hydrogen and carbon dioxide for methanol production, in accordance with embodiments of the present disclosure.

### DETAILED DESCRIPTION

In embodiments, the present disclosure is directed to a process of using Low-Temperature Methane Steam Reforming to efficiently produce “Green” Methanol. In embodiments, techniques may be performed in relation to Integrated Energy Systems (IESs), such as for use in green industrial processes that produce few or no carbon emissions, to capture carbon from an emission source, for resource production, and associated devices and methods. IESs of the present technology may include a power plant (e.g., a primary power plant) that is integrated with one or more industrial processes and resource production plants to provide power with few or no carbon emissions using excess power and/or steam from the power plant. The present disclosure includes systems and methods that may address many problems associated with conventional resource production processes, such as reducing carbon emissions and improving economic viability.

Methanol (CH₃OH) is a highly versatile chemical widely used for industrial purposes and prevalent in our everyday lives, additionally it is an energy-intensive source and sustainable fuel that could help in shaping the future economy. Methanol is a base material in the production of acetic acid and formaldehyde, and also increasingly being used in ethylene and propylene production. Methanol is one of the most prolific intermediate materials for the production of other chemicals and materials. In the chemical industry methanol mainly serves as a raw material in the production of formaldehyde, olefins, ethanol, acetic acid, MTBE, DME, as well as biodiesel. So, renewable methanol is a pre-requisite for making a broad range of chemical products “green” such as polymer fibers for the textile industry, plastics for packaging, glues, adsorbents/diapers, paints, adhesives, solvents, and much more.

Besides its use in the chemical, construction and plastics industries, methanol also serves as a fuel or fuel additive. For example, M85 consists of 85% Methanol and 15% Gasoline. For example, as fuel, Methanol can be used in the following ways: to fire rapid-start utility peak-shaving combustion turbines (peak-shaving is a strategy where utilities or large energy consumers reduce their electricity consumption during periods of highest demand, usually when electricity prices are most expensive); to substitute for or blend with gasoline to power vehicles; to be converted to gasoline; or to be converted to dimethyl ether (DME) to power diesel engines. Methanol is an extremely efficient hydrogen carrier because one methanol molecule has more hydrogen atoms than one hydrogen molecule. Methanol is a liquid at ambient conditions. Therefore, methanol can be handled, stored, and transported with ease by leveraging existing industrial infrastructures. The global methanol demand reached 107 million tons in 2021 and is expected to rise to approximately 500 million tons by 2050. This implies that more sustainable methanol manufacturing processes are needed in the near future. In addition, considering the global climate challenges, methanol is a potential alternative fuel to scale down the reliance on fossil fuels as a means of energy storage and transportation.

Conventionally, Methanol (CH₃OH) is produced from natural gas (Methane) using a steam reforming process to produce synthesis gas (syngas), a mixture of Hydrogen (H₂), Carbon Monoxide (CO), and Carbon Dioxide (CO₂), and a catalytic process to convert the syngas to Methanol (CH₃OH). Methane steam reforming (MSR) is the most common method of producing commercial bulk hydrogen to use in the production of Methanol. About 90% of methanol production is currently from natural gas, and other technologies are often not substituted on an industrial scale due to cost and undesirable efficiency. Currently, there is no existing process that can simultaneously produce all necessary gases for Methanol (CH₃OH) production and the required energy demand with few or no carbon emissions.

In broad terms, steam reforming is a process of producing hydrogen by combining steam and a hydrocarbon. In most cases, the hydrocarbon is Methane in the form of natural gas, because of its low cost and high availability. Steam and Methane are typically reacted in a reformer at temperatures>500° C. in the presence of a metal-based catalyst, for example a group 8 to 10 metal-based catalyst such as a nickel (Ni) based catalyst, to produce syngas. The overall methane steam reforming (MSR) reaction can be described by Equation 1:

CH₄+2H₂O↔CO₂+4H₂ (1)

Equation 1 is in fact composed of multiple reactions.

The first reaction, Equation 2, is methane reforming where Methane (CH₄) reacts with steam (H₂O) to produce Hydrogen (H₂) and Carbon Monoxide (CO):

CH₄+H₂O↔CO+3H₂ (2)

Equation 2 is strongly endothermic and typically is operated at 700-1000° C.

The second reaction, Equation 3, is the water gas shift (WGS) reaction where the produced Carbon Monoxide (CO) and steam (H₂O) also react to produce Carbon Dioxide (CO₂):

CO+H₂O↔CO₂+H₂ (3)

The reaction of Equation 3 is moderately exothermic and takes place between 200-450° C.

The complete methane steam reforming reaction is produced by adding Equations 2 and 3 to generate Equation 1:

CH₄+2H₂O↔CO₂+4H₂ (1)

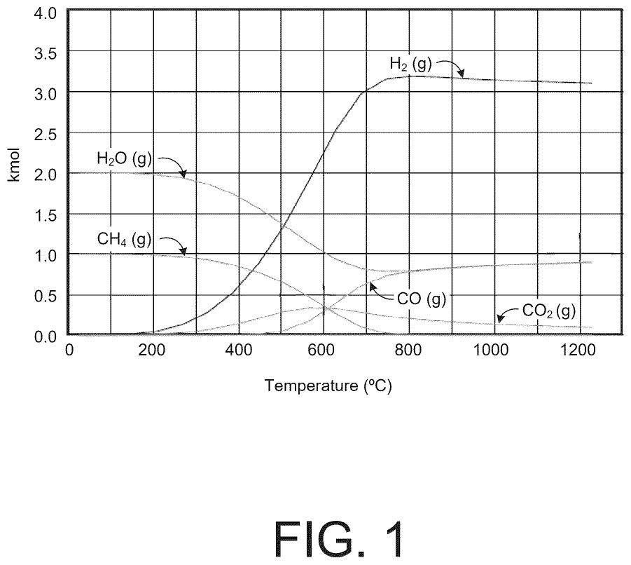

A typical thermodynamic representation of the reforming reactions in Equations 1-3 are shown in FIG. 1. As can be seen in FIG. 1, most Methane gas (CH₄) is completely converted at temperatures above 700° C. Conventional Methane steam reforming is operated at temperatures in the range of about 700-1000° C., and therefore a mixture of Hydrogen (H₂), Carbon Monoxide (CO), and Carbon Dioxide (CO₂) is produced, along with unreacted steam (H₂O). MSR is a highly endothermic process and, therefore, conventional methane steam reforming requires large energy input to maintain a temperature in a range of 700-1000° C. and a pressure of about 1-50 bar. Presently, fossil fuels such as natural gas, petroleum, and coal must be used to supply the required reformer heat demand. The current methane steam reforming process, therefore, results in significant Carbon Dioxide (CO₂) emissions into the atmosphere which is a concern for its environmental impact pertaining to green-house gas concentrations affecting global climate change. Environmental analysis shows that approximately 0.71 metric tons of CO₂ equivalent are emitted per metric ton of methanol produced when producing methanol using methane steam reforming.

The systems and methods of the present disclosure utilize low-temperature methane steam reforming to produce CO₂ and H₂ for the production of “green” Methanol, i.e. Methanol produced with few or no carbon emissions. As can be seen in FIG. 1, when the temperature of the methane steam reforming process is kept below 500° C., only Hydrogen (H₂) and Carbon Dioxide (CO₂) are produced while Carbon Monoxide (CO) production is suppressed. Therefore, a product stream including Hydrogen (H₂) and Carbon Dioxide (CO₂) for the production of Methanol can be produced at much lower temperatures than conventional methane steam reforming. Low-temperature methane steam reforming offers advantages over high temperature methane steam reforming such as: requiring less energy for the endothermic reaction which can enable the reformer to be preheated and/or powered by heat recovery units; reducing costs by enabling the use of less expensive materials for equipment (reactors, reformer tubes, etc.); allowing for quicker start-up; and eliminating the need for a CO shift reactor, leading to more economical on-site Carbon Dioxide (CO₂) and Hydrogen (H₂) production.

The Hydrogen (H₂) and Carbon Dioxide (CO₂) produced in a low-temperature (LT) methane steam reforming (MSR) process will exit the process in a combined gas stream with trace Carbon Monoxide (CO) as well as unreacted Methane gas (CH₄) and steam (H₂O). In a series of steps, these gases can be separated such that the Hydrogen (H₂), Carbon Dioxide (CO₂) and Carbon Monoxide (CO) can be used to produce Methanol and other useful products, the unreacted Methane gas (CH₄) can be recycled back to the low-temperature methane steam reforming process, and the steam (H₂O) can be condensed into to water to be recycled.

Initially, the combined gas stream exiting the low-temperature methane steam reforming process can be fed to a phase separator where the unreacted steam (H₂O) is condensed and separated the from the gas mixture to produce a ‘dry’ gas mixture. The dry gas mixture can then be fed into a separation unit, such as a Pressure Swing Adsorption (PSA) unit, for Hydrogen (H₂) separation. PSA is a gas separation technique that uses pressure changes to separate different gas species in a gas mixture. Since Hydrogen is the lightest gas in the dry gas mixture, it can easily be separated. The Hydrogen can then be used directly in a Methanol production process.

The remaining Methane gas (CH₄) can then be separated from the Carbon Dioxide (CO₂) and Carbon Monoxide (CO) if present, for example in a Cryogenic Fractional Distillation Column, to be recycled back to the low-temperature methane steam reforming process. The Cryogenic Fractional Distillation technique is a special distillation process designed to separate gases by utilizing low temperatures, for example less than −190° C. to less than −150° C., or less than −170° C. This method exploits the differences in boiling points of gases to achieve separation by condensation of the gas below its boiling point. Cryogenic Fractional Distillation can be performed in stages, and/or in separate or sequential distillation columns to separate multiple gases. In embodiments of the present disclosure, the Methane gas (CH₄) is partitioned out of the mixture and recycle back to the low-temperature (LT) methane steam reformer for re-use.

The Carbon Dioxide (CO₂) separated from the Methane gas (CH₄) can then be fed to a Solid Oxide Electrolytic Cell (SOEC). The process involves applying a voltage across the solid oxide electrolysis cell, causing the electrochemical reduction of Carbon Dioxide (CO₂) at the cathode to produce Carbon Monoxide (CO), and the simultaneous oxidation of Oxygen at the anode, generating O₂ gas. This process is often referred to as Carbon Dioxide (CO₂) electrolysis. The reacted Carbon Dioxide (CO₂) can be recycled back as part of the feed to the SOEC while the Carbon Monoxide (CO) can be fed directly to a methanol production process. The generated Oxygen (O₂) can be stored and/or sold.

In the methanol production process, Hydrogen (H₂) and Carbon Monoxide (CO) separated from the combined gas stream exiting the low-temperature methane steam reforming process can be combined to produce methanol following the reaction of Equation 4:

CO+2H₂↔CH₃OH (4)

The reaction of CO with H₂ is an exothermic reaction and typically is referred as CO Hydrogenation. The reaction can proceed at relatively low temperatures and pressures, such as a temperature between 20° and 300° C., for example about 250° C., and at a pressure between 5-10 MPa (50-100 bar), over a Nickel Oxide (NiO₂) catalyst or Copper-based catalyst (e.g., CuO/ZnO/Al₂O₃). This process is advantageous to the production of Methanol from syngas because it produces pure Methanol from a mixture of pure Carbon Monoxide (CO) and Hydrogen (H₂). Syngas is a mixture of Hydrogen (H₂), Carbon Monoxide (CO), and Carbon Dioxide (CO₂). Syngas may also contain water vapor, for example unreacted steam from methane stream reforming. When a syngas stream is reacted to produce Methanol, water is also produced both as the by-product of the Carbon Dioxide (CO₂) hydrogenation. The presence of water in conventional Methanol production processes using syngas leads to kinetic inhibition and acerated deactivation of the catalysts. Since there is no water present in either Hydrogen (H₂) or Carbon Monoxide (CO) gas stream of the Methanol production process of the present disclosure, and no Carbon Dioxide (CO₂) in the reaction, water will not be formed and therefore deactivation of the catalyst is eliminated. Methanol produced via this process can, therefore, be sustained and continuous without interruption to change the catalyst. This process is considered as a “green” methanol production technique because it does not produce Green-House-Gases (GHG) as comparing to typical High-Temperature Methane steam reforming.

### Exemplary Energy Requirements

To calculate the energy requirements for an embodiment of this disclosure, each step is presented including the estimates of the energy requirements. A Steam/Carbon ratio of 6 (mol/mol) is used as the starting material for the exemplary calculation. This ratio is represented as fresh natural gas and steam fed to the reformer at 78.4 metric tons per hour (t/hr) and 976 t/hr, respectively. In this exemplary analysis, the output gas mixture from a Low-Temperature MSR Reformer therefore results in 38.23 t/hr of H₂, 10.3 t/hr of CO, 197.7 t/hr of CO₂, 67.3 t/hr of CH₄, and 808.5 t/hr of steam (H2O). This output gas mixture is then fed into (1) one or more phase separator to condense the steam, (2) one or more PSA units to partition out H₂, (3) one or more cryogenic distillation columns to partition the gas mixture into three gas streams: CO₂ in one stream, CH₄ in one stream, and the third stream is CO, (4) the CO₂ stream is fed into a (5) SOEC stack to produce three separate gas streams: O₂, CO₂, and CO, and (6) CO hydrogenation to produce Methanol. In embodiments, CO from the cryogenic distillation columns and CO from the SOEC stack can be used in CO hydrogenation to produce Methanol. In embodiments, CO₂ from the SOEC stack can be recycled back to the SOEC.

For this analysis it was found that the total energy requirement for gas separation and Methanol Production is ~787 MWh, with a total Methanol produced is 124.74 t/hr. Therefore, the energy per ton of Methanol is 6.31 MWh/t of Methanol. Comparing this to prototypical methane steam reforming process that produces between 8.72-9.05 MWh/t of Methanol, the present exemplary embodiment has a better energy efficiency and with no GHG emission. In addition, there are values (market price) that can be leveraged with the co-products generated during the process such as O₂ (41.42 t/h); H₂ (38.23−15.6=22.63 t/hr); and CO₂ (98.85 t/hr). In sum, this exemplary embodiment demonstrates the uniqueness of the systems and methods of the present disclosure to produce “Green” Methanol plus other valuable co-products.

### Integrated Energy Systems

Pure Methanol (CH₃OH) produced by the systems and methods described herein, may be stored, sold, and/or used within an Integrated Energy System (IES) to produce other “green” chemical products. Co-products such as Oxygen (O₂), Hydrogen (H₂), Carbon Monoxide (CO), Carbon Dioxide (CO₂) can be used as feedstock along with the produced Methanol (CH₃OH) for the production of a range of important industrial chemicals. For example, three industrial products that use Methanol as feedstock are Formaldehyde, Ethanol, and Olefin.

Because of the drive toward cleaner and more efficient forms of power production, nuclear power will be increasingly important in the coming years. In operation, nuclear power plants use the nuclear fission process to generate heat, which is then used to produce steam to turn turbines and generate electricity. This process can result in the production of electrical power that reduces the need for coal and natural gas to produce electricity. Nuclear power plants provide reliable baseload power without emitting greenhouse gases such as Carbon Dioxide (CO₂) during operation, making them attractive for countries that are seeking to reduce carbon emissions and enhance energy security. Due to the advantages of nuclear energy for providing electricity, the present disclosure presents novel methods of using nuclear power in integrated energy systems for carbon capture and “green” resource production, such as the production of “green” chemical products.

In embodiments, the IES includes a power plant system having multiple small modular nuclear reactors (SMRs) specifically configured to operate in unison to support one or more of the industrial processes. SMRs are nuclear reactors that are smaller in terms of size (e.g., dimensions) and power compared to large, conventional nuclear reactors. Moreover, they are modular in that some or all of their systems and components can be factory-assembled and transported as a unit to a location for installation. In some aspects of the present technology, the multiple SMRs of the integrated energy system can flexibly and dynamically provide electricity, thermal, steam, or a combination of electricity, thermal, and steam to the industrial processes due to the modularity and flexibility of the SMRs. That is, a configuration of the SMRs can be switched during operation to provide varying levels of steam and electricity output depending on the operational states and/or demands of the industrial processes.

In embodiments, a power plant of the present disclosure can be a permanent or temporary installation built at or near (e.g., roughly 1 km from) the location of an industrial process facility or can be a mobile or partially mobile system that is moved to and assembled at or near the location of the industrial process facility. More generally, the power plant can be local (e.g., positioned at or near) to the industrial processes/operations it supports. For example, the power plant can be located within a threshold distance of 0.4 km (0.25 mile), within 0.8 km (0.5 mile), within 3.22 km (2 miles), within 4.82 km (3 miles), or within 8.1 km (5 miles) of the industrial processes/operations it supports. In embodiments, the power plant is configured to supply a portion of electricity to a power grid.

For example, an IES of the present disclosure may include a Low-Temperature Methane Steam Reforming (MSR) process for resource production and associated devices and methods. Resource production may include producing hydrogen gas (H₂) and carbon dioxide gas (CO₂) in a Low-Temperature MSR plant, separating the hydrogen gas (H₂) and carbon dioxide gas (CO₂) from the combined gas stream of the Low-Temperature MSR and recycling unreacted components back to the Low-Temperature MSR plant, producing and purifying carbon monoxide gas (CO) and oxygen gas (O₂) in an electrolysis plant, and producing methanol (CH₃OH) in a methanol synthesis reactor. In embodiments, the hydrogen (H₂) from the Low-Temperature MSR plant and the carbon monoxide (CO) from the electrolysis plant and from the cryogenic distillation column(s) may be used to produce methanol (CH₃OH) in the methanol synthesis reactor. In embodiments, the Carbon Dioxide (CO₂) from the Low-Temperature MSR plant may be used to produce the Carbon Monoxide (CO) and the Oxygen (O₂) in the electrolysis plant. Methanol (CH₃OH) produced in an IES of the present disclosure may be utilized along with co-products such as Oxygen (O₂), Hydrogen (H₂), Carbon Monoxide (CO), Carbon Dioxide (CO₂) as “green” chemical feedstock for the production of a range of important industrial chemicals. For example, three industrial products that use Methanol as feedstock are Formaldehyde, Ethanol, and Olefin. In embodiments, resources produced in an IES of the present disclosure may be recycled within the IES, removed from the IES, stored for future use, or sold.

An IES of the present disclosure can improve current Methanol (CH₃OH) and chemical production by producing low carbon electrical and thermal energy that can be used to power and/or heat all stages of the IES. An IES of the present disclosure may, therefore, may be an energy independent closed loop system that may produce Methanol and other useful resources from steam and a methane source (i.e., natural gas).

An IESs of the present disclosure can reduce and/or eliminate carbon emissions associated with typical syngas production via methane steam reforming by coupling low-temperature MSR to an emission free energy source, such as one or more small modular nuclear reactor (SMR) described herein. The low temperature MSR provides advantages over traditional high-temperature methane steam reforming. The low-temperature process requires less stringent equipment and operational control, which in a long run, results in lower operational costs and the equipment would be less expensive and will last much longer than current practices. In addition, replacement cost of catalysts due to de-activation due to water and higher temperature can also have substantial cost saving.

Not only can a small modular nuclear reactor (SMR) of the present disclosure produce emission free energy, but it can also generate super-heated process steam for industrial applications, see for example, Applicant's U.S. patent application Ser. No. 18/116,819, filed on Mar. 2, 2023, and entitled “Small Modular Nuclear Reactor Integrated Energy Systems for Energy Production and Green Industrial Applications” which is incorporated herein by reference in its entirety. An IES of the present disclosure therefore can suppress CO₂ emission during the methane steam reforming process, eliminate the use of natural gas an as energy source to support the entire production cycle, and provide super-heated process steam, such as super-heated steam, for the low-temperature MSR process.

Another unique aspect of IESs of the present disclosure is the utilization of an electrolysis process, for example a solid oxide electrolysis stack, such as a Solid Oxide Electrolysis Cell (SOEC) or a CO-Electrolysis Cell, to produce a combination of Carbon Monoxide (CO) and Carbon Dioxide (CO₂) gases for the production of “Green” Methanol without the use of another reforming processes to produce Carbon Monoxide (CO), see for example Applicant's U.S. patent application Ser. No. 18/486,971, filed on Oct. 13, 2023, and entitled “Small Modular Nuclear Reactor Integrated Energy Systems for In-Situ, On-Demand Hydrogen Generation and/or the Production of Sodium Formate,” which is incorporated herein by reference in its entirety. This setup eliminates additional carbon dioxide emission. A solid oxide electrolysis stack for converting CO₂ to CO, CO₂, and O₂ is found under World patent WO 2014/154253 A1 “A process for producing CO from CO, in a solid oxide electrolysis cell,” the disclosure of which is incorporated herein by reference in its entirety. The technology disclosed in this patent primarily utilizes a solid oxide electrolysis cell (SOEC) stack to achieve this conversion by applying an electric current to CO₂ on the fuel side, producing CO and releasing O₂ on the oxygen side.

Certain details are set forth in the following description and in FIGS. 1-10 to provide a thorough understanding of various embodiments of the present technology. In other instances, well-known structures, materials, operations, and/or systems often associated with nuclear reactors, power plant systems, integrated energy systems, chemical production plants, industrial process plants, electrolysis systems, direct air capture (DAC) plants, oil refineries, and the like, are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth. The terminology used below is to be interpreted in its broadest reasonable manner, even though it is being used in conjunction with a detailed description of certain examples of embodiments of the technology.

The accompanying Figures depict embodiments of the present technology and are not intended to limit its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the present technology. In addition, those of ordinary skill in the art will appreciate that further embodiments of the present technology can be practiced without several of the details described below.

Each of the references cited herein is incorporated herein by reference in its entirety. However, to the extent any materials incorporated herein by reference conflict with the present disclosure, the present disclosure controls. The headings provided herein are for convenience only and should not be construed as limiting the subject matter disclosed.

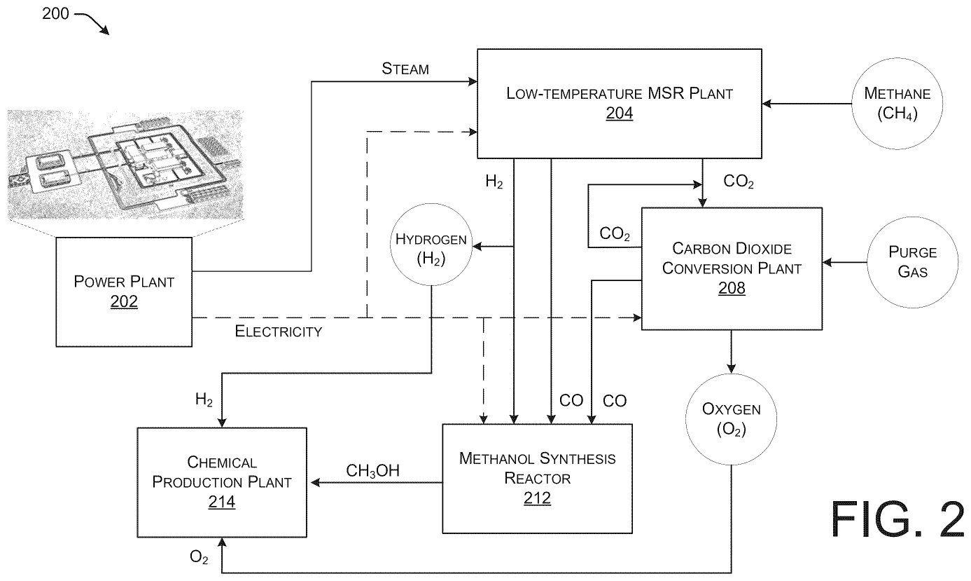

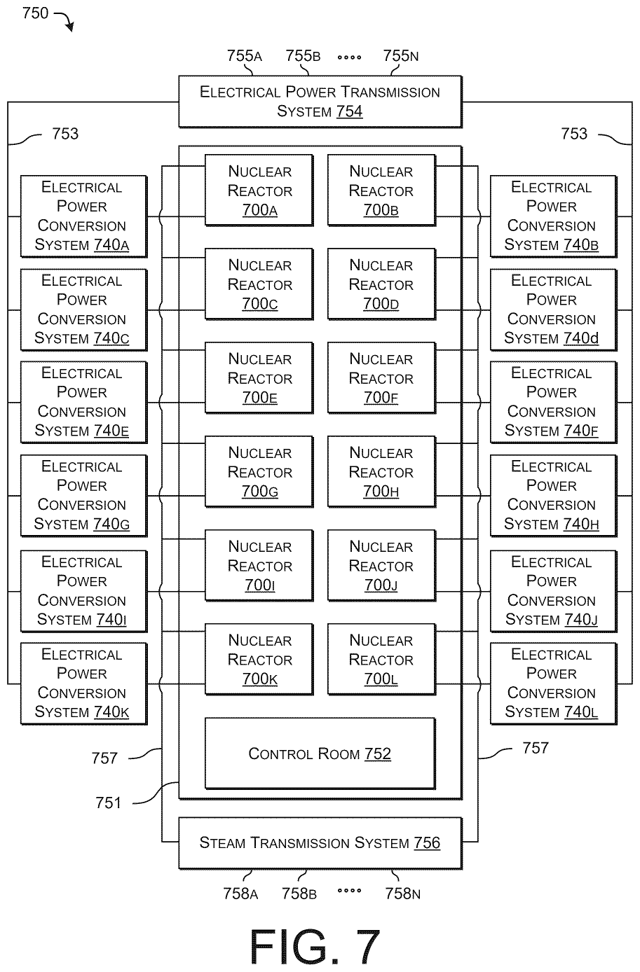

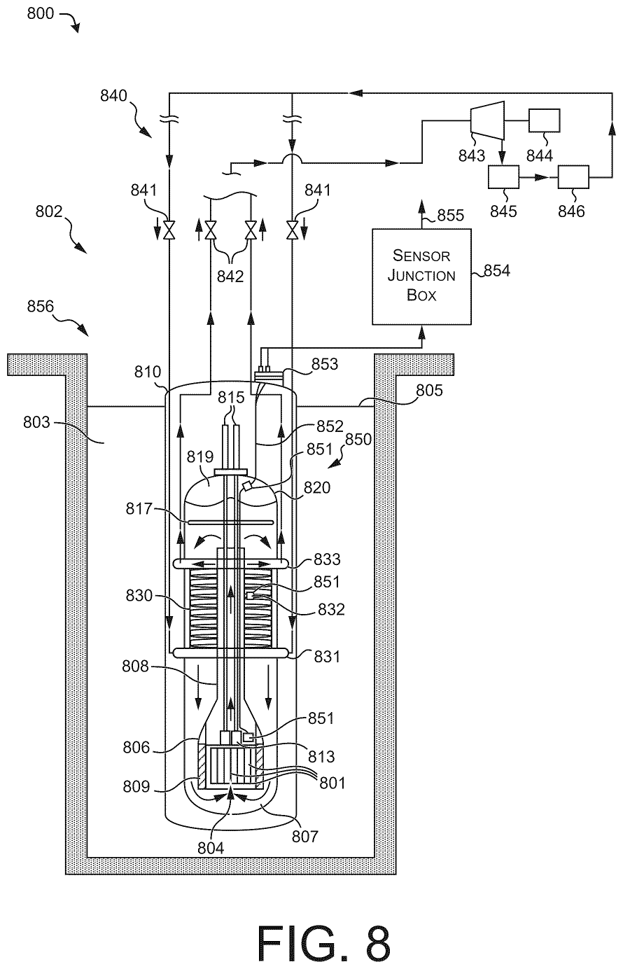

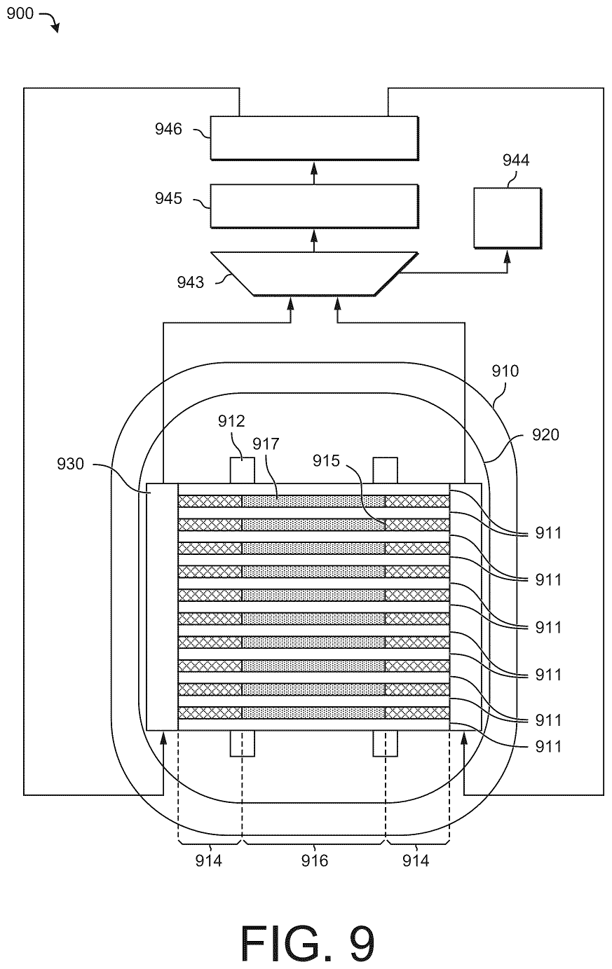

FIG. 2 schematically illustrates a representation of an integrated energy system (IES) 200 that includes a small modular nuclear reactor (SMR) system integrated with a Low-Temperature Methane Steam Reforming (MSR) process and Methanol (CH₃OH) production system. The IES 200 may include a Power Plant 202, a Low-Temperature MSR Plant 204, a Carbon Dioxide Conversion Plant 208, a Methanol Synthesis Reactor 212, and auxiliary equipment such as heaters, compressors, pumps, and control systems necessary to operate the IES 200 safely and at the desired conditions. In embodiments the power plant 202 may include the power plant system 750 of FIG. 7, in accordance with additional embodiments of the present technology. The power plant 202 may include a nuclear power module (NPM) including one or more light water nuclear reactor (LWR), one or more SMR, and any reactor system 800 of FIG. 8, reactor system 900 of FIG. 9, and system of nuclear reactors 700 of FIG. 7. In embodiments, the Power Plant 202 can be configured to provide electrical and/or thermal energy to the Low-Temperature MSR Plant 204, the Carbon Dioxide Conversion Plant 208, and the Methanol Synthesis Reactor 212.

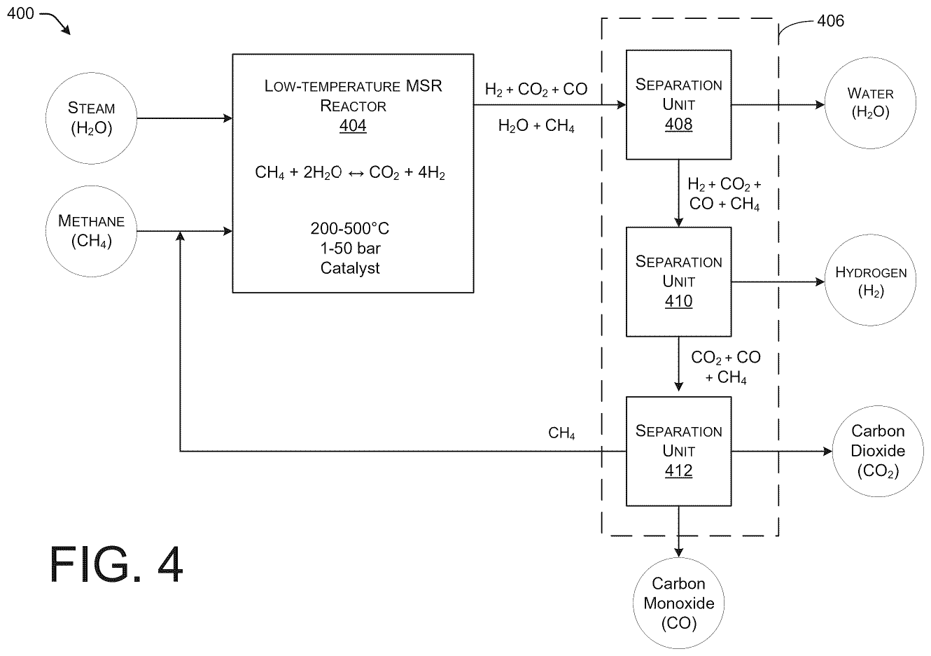

In the illustrated embodiment, the Power Plant 202 may be configured to provide steam, thermal energy, and/or electricity to the Low-Temperature MSR Plant 204. In embodiments, the Low-Temperature MSR Plant 204 may include the Low-Temperature MSR Plant 400 described in FIG. 4. In embodiments, the Low-Temperature MSR Plant 204 can be configured to receive Methane (CH₄) and steam (H₂O), and to produce Hydrogen (H₂) and Carbon dioxide (CO₂). In embodiments, Carbon Monoxide (CO) may also be produced in the Low-Temperature MSR Plant 204. In embodiments, the Methane (CH₄) may be received from a supplier, or maybe generated onsite. For example, the Methane (CH₄) may be obtained from purifying natural gas or other economical sources. In embodiments, the steam (H₂O) may include steam supplied directly from the Power Plant 202. In embodiments, the steam (H₂O) may include steam from a clean water source other than the Power Plant 202, for example clean water from a water treatment plant that has been heated and/or treated to the meet the desired operating conditions. In embodiments, the steam (H₂O) may be heated to the desired operating temperature using auxiliary heaters, such as electric heaters and/or heat exchangers. In embodiments, the steam (H₂O) may be compressed to the desired process pressure using auxiliary compressors or pumps. In embodiments, any auxiliary equipment within the IES 200, such as heaters, compressors, pumps, building lights, climate control, control systems, etc., can receive power from the Power Plant 202 and/or a power grid.

In embodiments, the Low-Temperature MSR Plant 204 can include one or more separation units configured to separate unreacted steam (H₂O), unreacted Methane (CH₄), the Hydrogen (H₂), the Carbon Dioxide (CO₂), and the Carbon Monoxide (CO) if present. In embodiments, the separated components from the Low-Temperature MSR Plant 204 may be recycled within the IES 200, used in downstream processes connected to the IES 200, removed from the IES 200, stored for future use, and/or sold. In embodiments, Low-Temperature MSR Plant 204 may include the Low-Temperature MSR Plant 400 described with reference to FIG. 4.

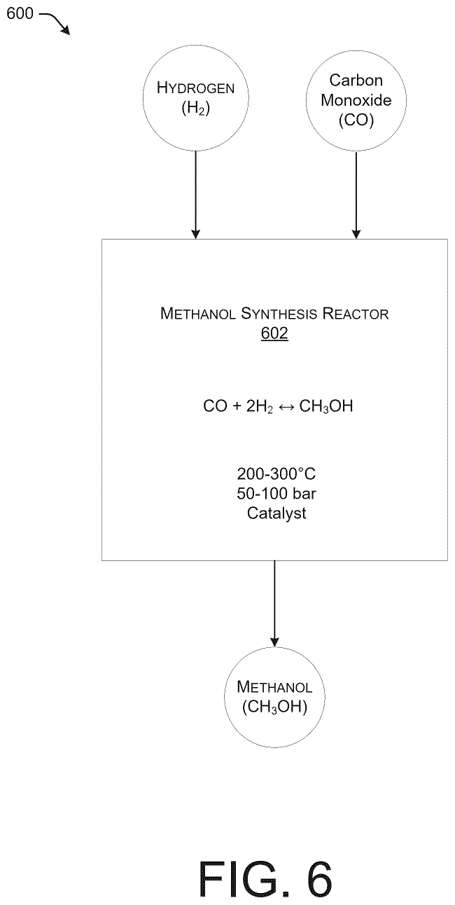

In embodiments, the Hydrogen (H₂) produced in the Low-Temperature MSR Plant 204 may be recycled within the IES 200, used in downstream processes connected to the IES 200, removed from the IES 200, stored for future use, and/or sold. For example, a first portion of the Hydrogen (H₂) can be removed from the IES 200 while a second portion of the Hydrogen (H₂) is fed to a downstream process, such as the Methanol Synthesis Reactor 212. In embodiments, the Carbon Monoxide (CO) produced in the Low-Temperature MSR Plant 204 may be fed to the Methanol Synthesis Reactor 212. In embodiments, the Methanol Synthesis Reactor 212 may include the Methanol Synthesis Reactor 602 described in FIG. 6.

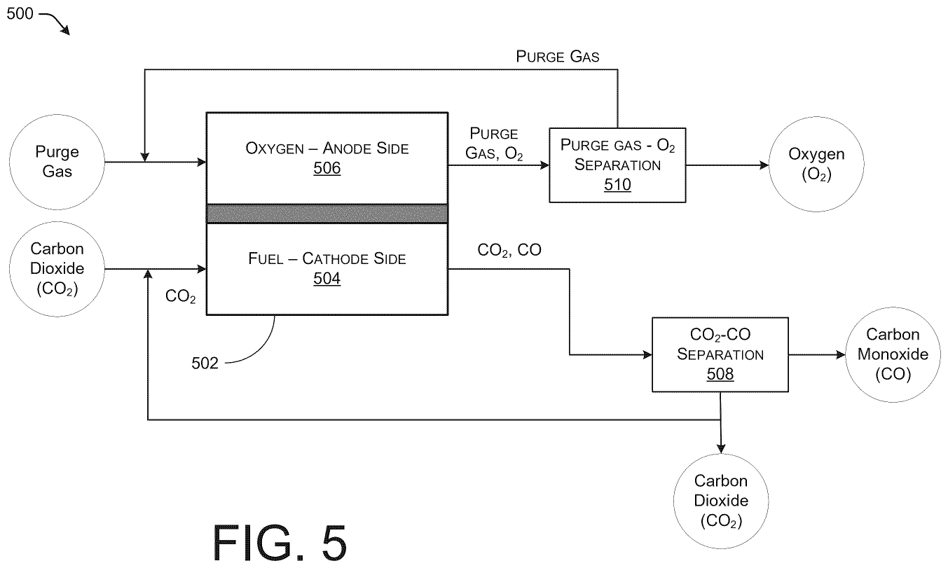

In embodiments, the Carbon Dioxide (CO₂) produced in the Low-Temperature MSR Plant 204 may be recycled within the IES 200, used in downstream processes connected to the IES 200, removed from the IES 200, stored for future use, and/or sold. For example, the Carbon Dioxide (CO₂) can be fed to the Carbon Dioxide Conversion Plant 208. In embodiments, the Carbon Dioxide Conversion Plant 208 may include the Carbon Dioxide (CO₂) Conversion Plant 500 described in FIG. 5. In embodiments, the Carbon Dioxide Conversion Plant 208 is configured to produce Carbon Monoxide (CO), Oxygen (O₂) and Carbon Dioxide (CO₂) from process inputs within the IES 200, for example, electricity from the Power Plant 202 and Carbon Dioxide (CO₂) from the Low-Temperature MSR Plant 204. In embodiments, the Carbon Dioxide (CO₂) produced in the Carbon Dioxide Conversion Plant 208 is configured to be recycled back to the input of the Carbon Dioxide Conversion Plant 208. In embodiments, the Carbon Dioxide Conversion Plant 208 is configured to produce Carbon Monoxide (CO) and Oxygen (O₂), and Carbon Dioxide (CO₂) from sources outside of the IES 200, such as purge gas and/or Carbon Dioxide (CO₂), for example purified Carbon Dioxide (CO₂) from an emission source.

In embodiments, the Methanol Synthesis Reactor 212 is configured to produce Methanol (CH₃OH) via Carbon Monoxide (CO) hydrogenation using inputs from within the IES 200, for example, electricity from the Power Plant 202, Hydrogen (H₂) from the Low-Temperature MSR Plant 204, and Carbon Monoxide (CO) from the Low-Temperature MSR Plant 204 and/or the Carbon Dioxide Conversion Plant 208. In embodiments, the Methanol produced within the Methanol Synthesis Reactor 212 may be used in downstream processes connected to the IES 200, such as a Chemical Production Plant 214, removed from the IES 200, stored for future use, and/or sold. In embodiments, intermediate streams, and products within the IES 200 may be recycled in order to optimize overall yield and efficiency. For example, the Chemical Production Plant 214 can be configured to receive one or more inputs from the IES 200 to produce one or more chemical products. For example, the Chemical Production plant 214 can include a process configured to receive Methanol (CH₃OH) and Oxygen (O₂) as feedstock for Formaldehyde (CH₃O) production, a process configured to receive Methanol (CH₃OH) and Hydrogen (H₂) as feedstock for Ethanol (CH₃CH₂OH) production, and a process configured to receive Methanol (CH₃OH) for Olefin production. In embodiments resources from outside the IES 200, such as from storage and/or from a supplier, may be added to any one or more process within the IES 200 in quantities sufficient to optimize overall yield and efficiency and to minimize byproduct and/or waste production.

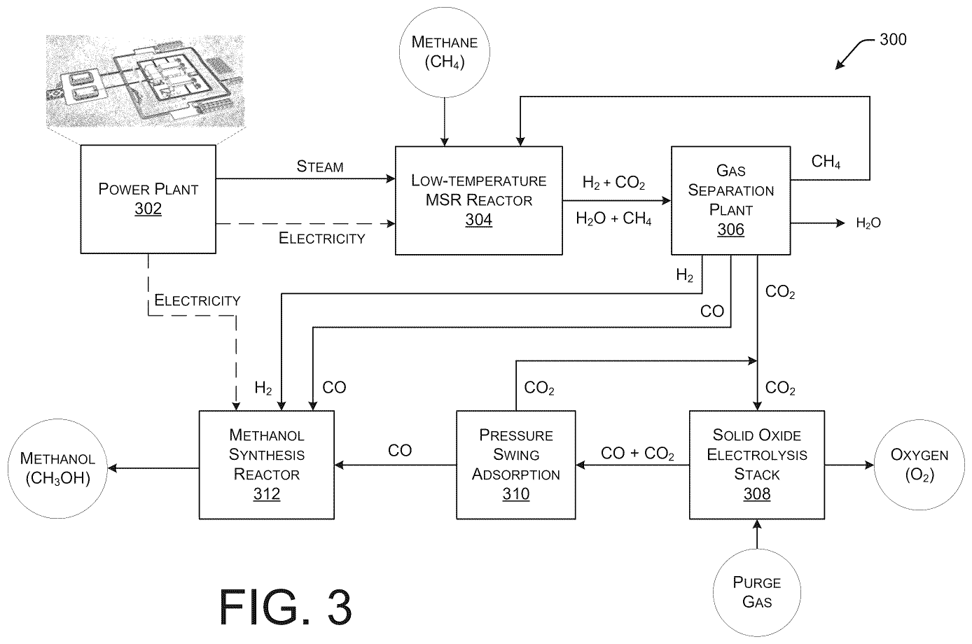

FIG. 3 schematically illustrates a representation of an integrated energy system (IES) 300 that includes a small modular nuclear reactor (SMR) system integrated with a Low-Temperature MSR process and Methanol (CH₃OH) production system. The IES 300 may include a Power Plant 302, a Low-Temperature MSR Reactor 304, a Gas Separation Plant 306, a Solid Oxide Electrolysis Stack 308, a Pressure Swing Adsorption (PSA) unit 310, a Methanol Synthesis Reactor 312, and auxiliary equipment such as heaters, compressors, pumps, and control systems necessary to operate the IES 300 safely and at the desired conditions. In embodiments the power plant 302 may include the power plant system 750 of FIG. 7, in accordance with additional embodiments of the present technology. The power plant 302 may include a nuclear power module (NPM) including one or more light water nuclear reactor (LWR), one or more SMR, and any reactor system 800 of FIG. 8, reactor system 900 of FIG. 9, and system of nuclear reactors 700 of FIG. 7. In embodiments, the Power Plant 302 can be configured to provide electrical, thermal energy and steam to the Low-Temperature MSR Reactor 304, the Gas Separation Plant 306, the Solid Oxide Electrolysis Stack 308, the Pressure Swing Adsorption (PSA) unit 310, and the Methanol Synthesis Reactor 312.

In the illustrated embodiment, the Power Plant 302 may be configured to provide steam, thermal energy, and electricity to the Low-Temperature MSR Reactor 304. In embodiments, the Low-Temperature MSR Reactor 304 may include the Low-Temperature MSR Reactor 404 described in FIG. 4. In embodiments, the Low-Temperature MSR Reactor 304 can be configured to receive Methane (CH₄) and steam (H₂O), and to produce Hydrogen (H₂) and Carbon dioxide (CO₂). In embodiments, Carbon Monoxide (CO) may also be produced in the Low-Temperature MSR Reactor 304. In embodiments, the Methane (CH₄) may be received from a supplier, or maybe generated onsite. For example, the Methane (CH₄) may be obtained from purifying natural gas or other economical sources. In embodiments, the steam (H₂O) may include steam supplied directly from the Power Plant 302. In embodiments, the steam (H₂O) may include steam from a clean water source other than the Power Plant 302, for example clean water from a water treatment plant that has been heated and/or treated to the meet the desired operating conditions. In embodiments, the steam (H₂O) may be heated to the desired operating temperature using auxiliary heaters, such as electric heaters and/or heat exchangers. In embodiments, the steam (H₂O) may be compressed to the desired process pressure using auxiliary compressors or pumps. In embodiments, any auxiliary equipment within the IES 300, such as heaters, compressors, pumps, building lights, climate control, control systems, etc., can receive power from the Power Plant 302 and/or a power grid.

In embodiments, the Hydrogen (H₂) and Carbon Dioxide (CO₂) produced in the Low-Temperature MSR Reactor 304, along with the unreacted steam (H₂O), unreacted Methane (CH₄), and trace amounts of Carbon Monoxide (CO), are output from the reactor in a single combined gas outlet stream. The combined gas outlet stream from the Low-Temperature MSR Reactor 304 can be fed to one or more separation units within the Gas Separation Plant 306, to separate the unreacted steam (H₂O), the unreacted Methane (CH₄), the Hydrogen (H₂), the Carbon Dioxide (CO₂), and the Carbon Monoxide (CO), if present, into purified gas streams. For example, the Gas Separation Plant 306 may be the Gas Separation Plant 406. The purified gas streams can then be recycled within the IES 300, used in downstream processes connected to the IES 300, removed from the IES 300, stored for future use, and/or sold. For example, the combined gas outlet steam can be separated such that the Hydrogen (H₂), Carbon Dioxide (CO₂) and Carbon Monoxide (CO) can be used to produce Methanol and other useful products, the unreacted Methane gas (CH₄) can be recycled back to the low-temperature methane steam reforming process, and the steam (H₂O) can be condensed into to water to be recycled or disposed of.

The Gas Separation Plant 306 can include a series of separation steps, for example the combined gas stream exiting the Low-Temperature MSR Reactor 304 can be fed to a phase separator where the unreacted steam (H₂O) is condensed and separated the from the gas mixture to produce a ‘dry’ gas mixture. The dry gas mixture can then be fed into a separation unit, such as a Pressure Swing Adsorption (PSA) unit, for Hydrogen (H₂) separation. PSA is a gas separation technique that uses pressure changes to separate different gas species in a gas mixture. Since Hydrogen is the lightest gas in the dry gas mixture, it can easily be separated. The Hydrogen can then be used directly in a Methanol production process. For example, a first portion of the Hydrogen (H₂) can be removed from the IES 300 while a second portion of the purified Hydrogen (H₂) is fed to a downstream process, such as the Methanol Synthesis Reactor 312. In embodiments, the Methanol Synthesis Reactor 312 may include the Methanol Synthesis Reactor 602 described in FIG. 6.

Within the Gas Separation Plant 306, the remaining Methane gas (CH₄) can then be separated from the Carbon Dioxide (CO₂) and Carbon Monoxide (CO), if present, for example in a Cryogenic Fractional Distillation Column. In embodiments, the Methane gas (CH₄) can be recycled back to the Low-Temperature MSR Reactor 304. In embodiments, the gas separation, such as Cryogenic Fractional Distillation, can be performed in stages, and/or in separate or sequential distillation columns to separate multiple gases. In the illustrated embodiment, the Methane gas (CH₄) is partitioned out of the mixture and recycled back to the Low-Temperature MSR Reactor 304 for re-use. In embodiments, the separated Carbon Monoxide (CO) can be fed to the Methanol Synthesis Reactor 312.

In embodiments, the purified Carbon Dioxide (CO₂) can be fed to the Solid Oxide Electrolysis Stack 308. In embodiments, the Solid Oxide Electrolysis Stack 308 may include the Solid Oxide Electrolysis Stack 502 described in FIG. 5. In embodiments, the Solid Oxide Electrolysis Stack 308 is configured to produce Carbon Dioxide (CO₂), Carbon Monoxide (CO) and Oxygen (O₂) from process inputs within the IES 300, for example, electricity from the Power Plant 302 and Carbon Dioxide (CO₂) from the Pressure Swing Adsorption unit 310. In embodiments, the Solid Oxide Electrolysis Stack 308 is configured to produce Carbon Dioxide (CO₂), Carbon Monoxide (CO) and Oxygen (O₂) from sources outside of the IES 300, such as purge gas and/or Carbon Dioxide (CO₂), for example purified Carbon Dioxide (CO₂) from an emission source.

In embodiments, the Carbon Monoxide (CO) produced in the Solid Oxide Electrolysis Stack 308 leaves the stack mixed with unconverted Carbon Dioxide (CO₂) in a single gas outlet stream. The mixed gas outlet stream from the Solid Oxide Electrolysis Stack 308 can be fed to a separation unit, such as the Pressure Swing Adsorption unit 310, to separate the Carbon Monoxide (CO) and Carbon Dioxide (CO₂) into two purified gas streams, for example a purified Carbon Monoxide (CO) stream and a purified Carbon Dioxide (CO₂) stream. In embodiments, the purified Carbon Monoxide (CO) stream and/or the purified Carbon Dioxide (CO₂) stream can be recycled within the IES 300, used in downstream processes connected to the IES 300, removed from the IES 300, stored for future use, and/or sold. In embodiments, the purified Carbon Monoxide (CO) stream can be fed to the Methanol Synthesis Reactor 312. In embodiments, the purified Carbon Dioxide (CO₂) stream can be recycled to the input of the Solid Oxide Electrolysis Stack 308.

In embodiments, the Methanol Synthesis Reactor 312 is configured to produce Methanol (CH₃OH) via Carbon Monoxide (CO) hydrogenation using inputs from within the IES 300, for example, electricity from the Power Plant 302, Hydrogen (H₂) from the Gas Separation Plant 306, and Carbon Monoxide (CO) from the Gas Separation Plant 306 and/or the Pressure Swing Adsorption unit 310. In embodiments, the Methanol produced within the Methanol Synthesis Reactor 312 may be, used in downstream processes connected to the IES 300, removed from the IES 300, stored for future use, and/or sold.

FIG. 4 is a schematic diagram of a Low-Temperature MSR Plant 400. In the illustrated embodiment, the Low-Temperature MSR Plant 400 includes one or more Low-Temperature MSR Reactor(s) 404, a Gas Separation Plant 406 including one or more separation units such as Separation Unit(s) 408, 410, and 412, and auxiliary equipment such as heaters, compressors, pumps, and control systems necessary to operate the Low-Temperature MSR Plant 400 safely and at the desired conditions. In embodiments, the Low-Temperature MSR Plant 400 may be configured to receive steam, thermal energy, and/or electricity from a power plant, such as a nuclear power module (NPM) including one or more light water nuclear reactor (LWR), one or more small modular nuclear reactor (SMR), and any reactor system 800 of FIG. 8, reactor system 900 of FIG. 9, and system of nuclear reactors 700 of FIG. 7.

In embodiments, the Low-Temperature MSR Reactor 404 can be configured to receive Methane (CH₄) and steam (H₂O), and to produce Hydrogen (H₂) and Carbon Dioxide (CO₂). In embodiments, Carbon Monoxide (CO) may also be produced in the Low-Temperature MSR Reactor 404. In embodiments, the Methane (CH₄) may be received from a supplier, or maybe generated onsite. For example, the Methane (CH₄) may be obtained from purifying natural gas or other economical sources. In embodiments, the steam (H₂O) can include steam supplied directly from a nuclear power plant, in accordance with embodiments of the present disclosure. In embodiments, the steam (H₂O) may include steam from a clean water source, for example clean water from a water treatment plant that has been heated and/or treated to the meet the desired operating conditions. In embodiments, the steam (H₂O) may be heated to the desired operating temperature using auxiliary heaters, such as electric heaters and/or heat exchangers. In embodiments, the steam (H₂O) may be compressed to the desired process pressure using auxiliary compressors or pumps. In embodiments, any auxiliary equipment within the Low-Temperature MSR Plant 400, such as heaters, compressors, pumps, building lights, climate control, control systems, etc., can receive power from a nuclear power plant in accordance with embodiments of the present disclosure, and/or a power grid.

In embodiments, Methane (CH₄) received within the Low-Temperature MSR Plant 400 can be reacted with steam (H₂O), for example in Low-Temperature MSR Reactor 404, to produce Hydrogen (H₂) and Carbon Dioxide (CO₂) according to Equation 1:

CH₄+2H₂O↔CO₂+4H₂ (1)

The Low-Temperature MSR process of the present disclosure is a process in which the operating temperature may be in the range of about 200-500° C., the pressure may be about 1-50 bar, and the reaction may take place in the presence of a catalyst, for example a group 8 to 10 metal-based catalyst such as a nickel (Ni) based catalyst and/or a Ruthenium (Ru) based catalyst. In embodiments, the feed composition to the Low-Temperature MSR Reactor 404 may include 10-100% Methane (CH₄) and 10-100% Steam (H₂O). In embodiments, the steam (H₂O) and the Methane (CH₄) may be fed to the Low-Temperature MSR Reactor 404 at a ratio of 6:1. In embodiments, thermal energy and/or power from a nuclear power plant in accordance with embodiments of the present disclosure can be directed to the Low-Temperature MSR Reactor 404 as needed to maintain the desired operating conditions.

In embodiments, the Hydrogen (H₂) and Carbon Dioxide (CO₂) produced in the Low-Temperature MSR Reactor 404, along with unreacted steam (H₂O), unreacted Methane (CH₄), and Carbon Monoxide (CO), if present, are output from the reactor in a single combined gas outlet stream. The combined gas outlet stream from the Low-Temperature MSR Reactor 404 can be fed to one or more separation units within a Gas Separation Plant 406, to separate the unreacted steam (H₂O), unreacted Methane (CH₄), Hydrogen (H₂) and Carbon Dioxide (CO₂) into purified gas streams. The purified gas streams can then be recycled within the Low-Temperature MSR Plant 400, used in downstream processes connected to the Low-Temperature MSR Plant 400, removed from Low-Temperature MSR Plant 400, stored for future use, and/or sold. For example, the combined gas outlet steam can be separated such that the Hydrogen (H₂), Carbon Dioxide (CO₂) and Carbon Monoxide (CO) can be used to produce Methanol and other useful products, the unreacted Methane gas (CH₄) can be recycled back to the low-temperature methane steam reforming process, and the steam (H₂O) can be condensed into to water to be recycled or disposed of.

The Gas Separation Plant 406 can include a series of Separation Units 408, 410, and 412. For example, the combined gas stream exiting the Low-Temperature MSR Reactor 404 can be fed to a Separation Unit 408, such as a phase separator, where the unreacted steam (H₂O) is condensed and separated the from the gas mixture to produce a ‘dry’ gas mixture of Hydrogen (H₂), Carbon dioxide (CO₂), Methane (CH₄), and Carbon Monoxide (CO) if present. The dry gas mixture can then be fed into a Separation Unit 410, such as a Pressure Swing Adsorption (PSA) unit, for Hydrogen (H₂) separation. PSA is a gas separation technique that uses pressure changes to separate different gas species in a gas mixture. Since Hydrogen (H₂) is the lightest gas in the dry gas mixture, it can easily be separated. The Hydrogen (H₂) can then be used directly in a Methanol production process. For example, a first portion of the Hydrogen (H₂) can be removed from the Low-Temperature MSR Plant 400 while a second portion of the purified Hydrogen (H₂) may be fed to a downstream process, such as a Methanol Synthesis Reactor 602 described in FIG. 6.

The remaining Methane gas (CH₄) can then be separated from the Carbon Dioxide (CO₂) and Carbon Monoxide (CO), if present, for example in a Separation Unit 412. In embodiments, the Separation unit 412 can include a Cryogenic Fractional Distillation Column. In embodiments, the Cryogenic Fractional Distillation can be performed in stages, and/or in separate or sequential distillation columns to separate multiple gases. The Carbon Dioxide (CO₂) separated from the Methane gas (CH₄) and Carbon Monoxide (CO) can then be fed to a Solid Oxide Electrolytic Cell (SOEC), such as the Solid Oxide Electrolysis Stack 502 described in FIG. 5. In embodiments, the Methane gas (CH₄) stream can be recycled back to the Low-Temperature MSR Reactor 404 for re-use. In embodiments, the Carbon Monoxide (CO) can be fed to a downstream process, such as a Methanol Synthesis Reactor 602 described in FIG. 6.

FIG. 5 is a block diagram of a Carbon Dioxide (CO₂) Conversion Plant 500, in accordance with embodiments of the present disclosure. In embodiments, the Carbon Dioxide (CO₂) Conversion Plant 500 may include one or more Solid Oxide Electrolysis Stack 502, a CO₂—CO Separation process 508, a Purge Gas-O₂ Separation process 510, and auxiliary equipment such as heaters, compressors, pumps, and control systems necessary to operate the Carbon Dioxide (CO₂) Conversion Plant 500 safely and at the desired conditions. In embodiments, the Carbon Dioxide (CO₂) Conversion Plant 500 may be configured to receive thermal energy and/or electricity from a power plant, such as a nuclear power module (NPM) including one or more light water nuclear reactor (LWR), one or more small modular nuclear reactor (SMR), and any reactor system 800 of FIG. 8, reactor system 900 of FIG. 9, and system of nuclear reactors 700 of FIG. 7, and other inputs (e.g., CO₂) from within or external to the IES.

A Solid Oxide Electrolysis Stack 502 of the embodiments may be configured to perform the electrochemical Carbon Dioxide (CO₂) reduction for Carbon Monoxide (CO) and Oxygen (O₂). In embodiments, the Solid Oxide Electrolysis Stack 502 may include a solid ceramic material, for example stabilized zirconia, such as yttria-stabilized zirconia (YSZ, a solid solution of Y₂O₃ and ZrO₂) and scandia-stabilized zirconia (ScSZ), gadolinia-doped ceria (abbreviated either as GDC) or Samaria-doped ceria (SDC). The Solid Oxide Electrolysis Stack 502 may include one or more of a Solid Oxide Electrolysis Cell (SOEC) or a Co-Electrolysis cell configured to produce Carbon Monoxide (CO) and Oxygen (O₂).

In embodiments, Carbon Dioxide (CO₂) is fed to the Solid Oxide Electrolysis Stack 502. In embodiments, the Carbon Dioxide (CO₂) may be fed to the Solid Oxide Electrolysis Stack 502 from storage, from a supplier, and/or directly from an upstream process. In embodiments, the Carbon Dioxide (CO₂) is purified Carbon Dioxide (CO₂) from a Low-Temperature MSR process, such as the Low-Temperature MSR Plant 500, and/or purified Carbon Dioxide (CO₂) from an emission source.

In embodiments, Carbon Dioxide (CO₂) is fed to the fuel side 504, i.e., the cathode side, of the Solid Oxide Electrolysis Stack 502 with an applied current and Oxygen (O₂) is generated. The equation that governs the Carbon Dioxide (CO₂) reduction for Carbon Monoxide (CO) and Oxygen (O₂) production is shown in Equation 5:

4CO₂→2CO₂+C2O+O₂ (5)

Note the molar ratio of Carbon Dioxide (CO₂) gas and Carbon Monoxide (CO) gas in Equation 5 is 1:1. Oxygen (O₂) generated in the reaction is then transported to the oxygen side 506, i.e., the anode side, of the Solid Oxide Electrolysis Stack 502. In embodiments, a purge gas, such as Carbon Dioxide (CO₂), air, or nitrogen may be used to flush the oxygen side 506 of the Solid Oxide Electrolysis Stack 502. In embodiments, Carbon Dioxide (CO₂) can be used to flush the oxygen side 506, instead of air, to mitigate the leakage of undesired gases, such as Nitrogen (N₂), into the fuel side 504 of the Solid Oxide Electrolysis Stack 502. Flushing the oxygen side 506 of the Solid Oxide Electrolysis Stack 502 with Carbon Dioxide (CO₂) gas has two advantages, more specifically (1) enhancing the oxygen production concentration and (2) providing means for feeding energy into the Solid Oxide Electrolysis Stack 502.

In embodiments, the Solid Oxide Electrolysis Stack 502 is operated at elevated temperatures (e.g., ~600° C.). Inlet gas to the fuel side 504 and/or flush gas to the oxygen side 506 may be heated, for example in one or more auxiliary heaters, prior to entering the Solid Oxide Electrolysis Stack 502. In embodiments, Joule heat, i.e., the heat produced when current is passed through the Solid Oxide Electrolysis Stack 502, may supply some or all of the heat necessary for the Solid Oxide Electrolysis Stack 502. In embodiments, Joule heat, auxiliary heaters, and/or means of heating known in the art may be used in combination to provide optimum operating conditions for the Solid Oxide Electrolysis Stack 502. In embodiments, the Carbon Dioxide (CO₂) Conversion Plant 500 may receive power and/or thermal energy from a nuclear power plant in accordance with embodiments of the present disclosure.

In embodiments, the product stream from the fuel side 504 of the Solid Oxide Electrolysis Stack 502, for example a gas stream containing Carbon Monoxide (CO) mixed with Carbon Dioxide (CO₂), may then be passed through a CO₂—CO Separation process 508 to separate the Carbon Monoxide (CO) and the Carbon Dioxide (CO₂). In embodiments, the CO₂—CO Separation process 508 may include one or more separation units, such as pressure swing adsorption (PSA), temperature swing adsorption (TSA), membrane separation, and cryogenic separation technology. Carbon Dioxide (CO₂) from the CO₂—CO Separation process 508 may be recycled within the Carbon Dioxide (CO₂) Conversion Plant 500, for example to the inlet of the Solid Oxide Electrolysis Stack 502, and/or used in downstream processes, such as in a Methanol (CH₃OH) production process. In embodiments, Carbon Monoxide (CO) from the CO₂—CO Separation process 508 may be recycled within the Carbon Dioxide (CO₂) Conversion Plant 500 and/or used in downstream processes, such as in a Methanol (CH₃OH) production process.

In embodiments, the product stream from the oxygen side 506 of the Solid Oxide Electrolysis Stack 502, for example a gas stream containing Oxygen (O₂) mixed with purge gas, may then be passed through a Purge Gas-O₂ Separation process 510 to separate the Oxygen (O₂) and the purge gas. In embodiments, the Purge Gas-O₂ Separation process 510 may include one or more separation units, such as PSA, adsorption, membrane separation, or any separation process sufficient to separate Oxygen (O₂) from the Purge Gas. In embodiments, the Purge gas from the Purge Gas-O₂ Separation process 510 may be recycled within the Carbon Dioxide (CO₂) Conversion Plant 500, for example to the inlet of the Solid Oxide Electrolysis Stack 502. Oxygen (O₂) from the Purge Gas-O₂ Separation process 510 may be used in downstream processes, further purified, stored, and/or sold, for example to hospitals and industry.

FIG. 6 is a schematic diagram of a Methanol Production Plant 600. In the illustrated embodiment, the Methanol Production Plant 600 includes one or more Methanol Synthesis Reactor(s) 602 and auxiliary equipment such as heaters, compressors, pumps, and control systems necessary to operate the Methanol Production Plant 600 safely and at the desired conditions. In embodiments, the Methanol Production Plant 600 may be configured to receive steam, thermal energy, and/or electricity from a power plant, such as a nuclear power module (NPM) including one or more light water nuclear reactor (LWR), one or more small modular nuclear reactor (SMR), and any reactor system 800 of FIG. 8, reactor system 900 of FIG. 9, and system of nuclear reactors 700 of FIG. 7, and other inputs (e.g., CO and H₂) from within or external to the IES.

In embodiments, the Methanol Synthesis Reactor 602 can be configured to receive Hydrogen (H₂) and Carbon Monoxide (CO), and to produce Methanol (CH₃OH). In embodiments, the Methanol Synthesis Reactor 602 is configured to produce Methanol (CH₃OH) via Carbon Monoxide (CO) hydrogenation over a catalyst, such as a Nickle Oxide (NiO₂) catalyst, and/or a Copper (Cu) and zinc oxide (ZnO) based catalyst (Cu/ZnO), at a temperature between 20° and 300° C., for example about 250° C., and at a pressure between 5-10 MPa (50-100 bar). The reaction is shown in Equation 4:

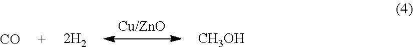

In embodiments, the Methanol Synthesis Reactor 602 receives purified Hydrogen (H₂) from a source such as the Low-Temperature MSR Plant 400, and purified Carbon Monoxide (CO) from a source such as the Carbon Dioxide (CO₂) Conversion Plant 500 and Separation Unit 412. In embodiments, the Methanol Synthesis Reactor 602 can be configured to receive purified Hydrogen (H₂) and/or Carbon Monoxide (CO) from a supplier.

There is significant advantage to the production of Methanol (CH₃OH) via Carbon Monoxide (CO) hydrogenation as described herein. By introducing purified Hydrogen (H₂) and purified Carbon Monoxide (CO) to the Methanol Synthesis Reactor 602 there is no water present in the process, and the hydrogenation process can take place at low-temperature. Because of the low temperature, the reaction can be sustained and continuous without interruption and additional cost to change out deactivated catalyst. This advantage and the improved efficiency of this process described herein contributes to the production of “green” Methanol (CH₃OH).

In embodiments, the Methanol (CH₃OH) produced within the Methanol Synthesis Reactor 602 may be used in downstream processes connected to the Methanol Synthesis Plant 600, removed from the Methanol Synthesis Plant 600, stored for future use, and/or sold.

FIG. 7 is a schematic view of a nuclear power plant system 750 (“power plant system 750”) including multiple nuclear reactors 700 (individually identified as first through twelfth nuclear reactors 700A-L, respectively) in accordance with embodiments of the present technology. The power plant system 750 can be a permanent or temporary installation built at or near (e.g., roughly 1 km from) the location of an industrial process facility or can be a mobile or partially mobile system that is moved to and assembled at or near the location of the industrial process facility. More generally, the power plant can be local (e.g., positioned at or near) to the industrial processes/operations it supports. For example, the power plant can be located within a threshold distance of 0.4 km (0.25 mile), within 0.8 km (0.5 mile), within 3.22 km (2 miles), within 4.82 km (3 miles), or within 8.1 km (5 miles) of the industrial processes/operations it supports. In embodiments, the power plant system 750 is configured to supply a portion of electricity to a power grid.

In embodiments, the power plant system 750 may produce and deliver electrical power to the power grid during peak times or anytime that there is a demand for energy production. For example, when consumer energy demand imposes a high energy demand on the power grid (“peak times”), the power plant system 750 may be configured to produce and provide the energy necessary for the power grid to meet the high consumer demand during peak times. In embodiments, the power plant system 750 may be configured to provide energy directly to the power grid as required to meet energy demand due to factors other than increased energy demand during peak times (e.g., an energy producing plant that provides energy to the power grid may be offline and unable to provide energy, which creates an increased energy production demand without an increased demand for consumer electrical power).

Each of the nuclear reactors 700 can be similar to, or identical to, the nuclear reactor system 800 and/or the nuclear reactor system 900 described in detail below with reference to FIG. 8 and FIG. 9. The power plant system 750 can be “modular” in that each of the nuclear reactors 700 can be operated separately to provide an output, such as electricity or steam. In embodiments, the power plant system may include Small Modular Reactors (SMRs), a microreactor, or other types of advanced reactors. In one embodiment, the nuclear reactor module may be a light pressurized water reactor (PWR) but may be different types of reactors utilizing a variety of fuels and located on terrestrial, maritime, and extraterrestrial sites. The power plant system 750 can include fewer than twelve of the nuclear reactors 700 (e.g., two, three, four, five, six, seven, eight, nine, ten, or eleven of the nuclear reactors 700), or more than twelve of the nuclear reactors 700. The power plant system 750 can be a permanent installation or can be mobile (e.g., mounted on a truck, tractor, mobile platform, and/or the like). In the illustrated embodiment, each of the nuclear reactors 700 can be positioned within a common housing 751, such as a reactor plant building, and controlled and/or monitored via a control room 752.

Each of the nuclear reactors 700 can be coupled to a corresponding electrical power conversion system 740 (individually identified as first through twelfth electrical power conversion systems 740A-L, respectively). The electrical power conversion systems 740 can include one or more devices that generate electrical power or some other form of usable power from steam generated by the nuclear reactors 700. For example, the electrical power conversion systems 740 can include features that are similar or identical to the power conversion system 840 described in detail below with reference to FIG. 8. In some embodiments, multiple ones of the nuclear reactors 700 can be coupled to the same one of the electrical power conversion systems 740 and/or one or more of the nuclear reactors 700 can be coupled to multiple ones of the electrical power conversion systems 740 such that there is not a one-to-one correspondence between the nuclear reactors 700 and the electrical power conversion systems 740.

The electrical power conversion systems 740 can be further coupled to an electrical power transmission system 754 via, for example, an electrical power bus 753. The electrical power transmission system 754 and/or the electrical power bus 753 can include one or more transmission lines, transformers, and/or the like for regulating the current, voltage, and/or other characteristic(s) of the electricity generated by the electrical power conversion systems 740. The electrical power transmission system 754 can route electricity via a plurality of electrical output paths 755 (individually identified as electrical output paths 755A-N) to one or more end users and/or end uses, such as different electrical loads of an integrated energy system as described in greater detail herein.

The power plant system 750 can be configured in a first operating state to provide electricity to a water treatment plant (e.g., via one or more of the electrical output paths 755 from the electrical power transmission system 754). The water treatment plant can route the produced high-quality water to the power plant system 750, and the power plant system 750 can use the water to produce high-quality steam. For example, the produced water can be used as a secondary coolant in a steam generator of one or more of the nuclear reactors 700. In some embodiments, the water treatment plant can be omitted and the power plant system 750 can utilize water from other sources to generate steam.

Each of the nuclear reactors 700 can further be coupled to a steam transmission system 756 via, for example, a steam bus 757. The steam bus 757 can route steam generated from the nuclear reactors 700 to the steam transmission system 756 which in turn can route the steam via a plurality of steam output paths 758 (individually identified as steam output paths 758A-N) to one or more end users and/or end uses, such as different steam inputs of an integrated energy system as described in greater detail below.

In some embodiments, the nuclear reactors 700 can be individually controlled (e.g., via the control room 752) to provide steam to the steam transmission system 756 and/or steam to the corresponding one of the electrical power conversion systems 740 to provide electricity to the electrical power transmission system 754. In some embodiments, the nuclear reactors 700 are configured to provide steam either to the steam bus 757 or to the corresponding one of the electrical power conversion systems 740 and can be rapidly and efficiently switched between providing steam to either. Accordingly, in some aspects of the present technology the nuclear reactors 700 can be modularly and flexibly controlled such that the power plant system 750 can provide differing levels/amounts of electricity via the electrical power transmission system 754 and/or steam via the steam transmission system 756. For example, where the power plant system 750 is used to provide electricity and steam to one or more industrial process—such as various components of the integrated energy systems described in the detail below—the nuclear reactors 700 can be controlled to meet the differing electricity and steam requirements of the industrial processes.

As one example, during a first operational state of an integrated energy system employing the power plant system 750, a first subset of the nuclear reactors 700 (e.g., the first through sixth nuclear reactors 700A-F) can be configured to provide steam to the steam transmission system 756 for use in the first operational state of the integrated energy system, while a second subset of the nuclear reactors 700 (e.g., the seventh through twelfth nuclear reactors 700G-L) can be configured to provide steam to the corresponding ones of the electrical power conversion systems 740 (e.g., the seventh through twelfth electrical power conversion systems 740G-L) to generate electricity for the first operational state of the integrated energy system. Then, during a second operational state of the integrated energy system when a different (e.g., greater or lesser) amount of steam and/or electricity is required, some or all the first subset of the nuclear reactors 700 can be switched to provide steam to the corresponding ones of the electrical power conversion systems 740 (e.g., the seventh through twelfth electrical power conversion systems 740G-L) and/or some or all of the second subset of the nuclear reactors 700 can be switched to provide steam to the steam transmission system 756 to vary the amount of steam and electricity produced to match the requirements/demands of the second operational state. Other variations of steam and electricity generation are possible based on the needs of the integrated energy system. That is, the nuclear reactors 700 can be dynamically/flexibly controlled during other operational states of an integrated energy system to meet the steam and electricity requirements of the operational state.

In contrast, some conventional nuclear power plant systems can typically generate a fixed amount of either steam or electricity for output, and cannot be modularly controlled to provide varying levels of steam and electricity for output. Moreover, it is typically difficult (e.g., expensive, time consuming, etc.) to switch between steam generation and electricity generation in conventional nuclear power plant systems. Specifically, for example, it is typically extremely time consuming to switch between steam generation and electricity generation in prototypical large nuclear power plant systems.

The nuclear reactors 700 can be individually controlled via one or more operators and/or via a computer system. Accordingly, many embodiments of the technology described herein may take the form of computer- or machine- or controller-executable instructions, including routines executed by a programmable computer or controller. Those skilled in the relevant art will appreciate that the technology can be practiced on computer/controller systems other than those shown and described herein. The technology can be embodied in a special-purpose computer, controller or data processor that is specifically programmed, configured or constructed to perform one or more of the computer-executable instructions described below. Accordingly, the terms “computer” and “controller” as generally used herein refer to any data processor and can include Internet appliances and hand-held devices (including palm-top computers, wearable computers, cellular or mobile phones, multi-processor systems, processor-based or programmable consumer electronics, network computers, micro-computers and the like). Information handled by these computers can be presented at any suitable display medium, including a liquid crystal display (LCD).

The technology can also be practiced in distributed environments, where tasks or modules are performed by remote processing devices that are linked through a communications network. In a distributed computing environment, program modules or subroutines may be located in local and remote memory storage devices. Aspects of the technology described herein may be stored or distributed on computer-readable media, including magnetic or optically readable or removable computer disks, as well as distributed electronically over networks. Data structures and transmissions of data particular to aspects of the technology are also encompassed within the scope of the embodiments of the technology.

FIG. 8 and FIG. 9 illustrate representative nuclear reactors that may be included in embodiments of the present technology. The nuclear reactor module of FIG. 8 or FIG. 9 may be a small modular reactor (SMR), a microreactor, or other types of advanced reactors. In one embodiment, the nuclear reactor module may be a light pressurized water reactor (PWR) but may be different types of reactors utilizing a variety of fuels and located on terrestrial, maritime, and extraterrestrial sites. FIG. 8 is a partially schematic, partially cross-sectional view of a nuclear reactor system 800 configured in accordance with embodiments of the present technology. The system 800 can include a power module 802 having a reactor core 804 in which a controlled nuclear reaction takes place. Accordingly, the reactor core 804 can include one or more fuel assemblies 801. The fuel assemblies 801 can include fissile and/or other suitable materials. Heat from the reaction generates steam at a steam generator 830, which directs the steam to a power conversion system 840. The power conversion system 840 generates electrical power, and/or provides other useful outputs, such as super-heated steam. A sensor system 850 is used to monitor the operation of the power module 802 and/or other system components. The data obtained from the sensor system 850 can be used in real time to control the power module 802, and/or can be used to update the design of the power module 802 and/or other system components.

The power module 802 includes a containment vessel 810 (e.g., a radiation shield vessel, or a radiation shield container) that houses/encloses a reactor vessel 820 (e.g., a reactor pressure vessel, or a reactor pressure container), which in turn houses the reactor core 804. The containment vessel 810 can be housed in a power module bay 856. The power module bay 856 can contain a cooling pool 803 filled with water and/or another suitable cooling liquid. The bulk of the power module 802 can be positioned below a surface 805 of the cooling pool 803. Accordingly, the cooling pool 803 can operate as a thermal sink, for example, in the event of a system malfunction.

A volume between the reactor vessel 820 and the containment vessel 810 can be partially or completely evacuated to reduce heat transfer from the reactor vessel 820 to the surrounding environment (e.g., to the cooling pool 803). However, in other embodiments the volume between the reactor vessel 820 and the containment vessel 810 can be at least partially filled with a gas and/or a liquid that increases heat transfer between the reactor vessel 820 and the containment vessel 810. For example, the volume between the reactor vessel 820 and the containment vessel 810 can be at least partially filled (e.g., flooded with the primary coolant 807) during an emergency operation.

Within the reactor vessel 820, a primary coolant 807 conveys heat from the reactor core 804 to the steam generator 830. For example, as illustrated by arrows located within the reactor vessel 820, the primary coolant 807 is heated at the reactor core 804 toward the bottom of the reactor vessel 820. The heated primary coolant 807 (e.g., water with or without additives) rises from the reactor core 804 through a core shroud 806 and to a riser tube 808. The hot, buoyant primary coolant 807 continues to rise through the riser tube 808, then exits the riser tube 808 and passes downwardly through the steam generator 830. The steam generator 830 includes a multitude of conduits 832 that are arranged circumferentially around the riser tube 808, for example, in a helical pattern, as is shown schematically in FIG. 8. The descending primary coolant 807 transfers heat to a secondary coolant (e.g., water) within the conduits 832, and descends to the bottom of the reactor vessel 820 where the cycle begins again. The cycle can be driven by the changes in the buoyancy of the primary coolant 807, thus reducing or eliminating the need for pumps to move the primary coolant 807.

The steam generator 830 can include a feedwater header 831 at which the incoming secondary coolant enters the steam generator conduits 832. The secondary coolant rises through the conduits 832, converts to vapor (e.g., steam), and is collected at a steam header 833. The steam exits the steam header 833 and is directed to the power conversion system 840.

The power conversion system 840 can include one or more steam valves 842 that regulate the passage of high pressure, high temperature steam from the steam generator 830 to a steam turbine 843. The steam turbine 843 converts the thermal energy of the steam to electricity via a generator 844. The low-pressure steam exiting the turbine 843 is condensed at a condenser 845, and then directed (e.g., via a pump 846) to one or more feedwater valves 841. The feedwater valves 841 control the rate at which the feedwater re-enters the steam generator 830 via the feedwater header 831. In other embodiments, the steam from the steam generator 830 can be routed for direct use in an industrial process, such as a hydrogen and oxygen production plant, a chemical production plant, and/or the like, as described in detail in this application. Accordingly, steam exiting the steam generator 830 can bypass the power conversion system 840.

The power module 802 includes multiple control systems and associated sensors. For example, the power module 802 can include a hollow cylindrical reflector 809 that directs neutrons back into the reactor core 804 to further the nuclear reaction taking place therein. Control rods 813 are used to modulate the nuclear reaction and are driven via fuel rod drivers 815. The pressure within the reactor vessel 820 can be controlled via a pressurizer plate 817 (which can also serve to direct the primary coolant 807 downwardly through the steam generator 830) by controlling the pressure in a pressurizing volume 819 positioned above the pressurizer plate 817.

The sensor system 850 can include one or more sensors 851 positioned at a variety of locations within the power module 802 and/or elsewhere, for example, to identify operating parameter values and/or changes in parameter values. The data collected by the sensor system 850 can then be used to control the operation of the system 800, and/or to generate design changes for the system 800. For sensors positioned within the containment vessel 810, a sensor link 852 directs data from the sensors to a flange 853 (at which the sensor link 852 exits the containment vessel 810) and directs data to a sensor junction box 854. From there, the sensor data can be routed to one or more controllers and/or other data systems via a data bus 855.

FIG. 9 is a partially schematic, partially cross-sectional view of a nuclear reactor system 900 (“system 900”) configured in accordance with additional embodiments of the present technology. In some embodiments, the system 900 can include some features that are at least generally similar in structure and function, or identical in structure and function, to the corresponding features of the system 800 described in detail above with reference to FIG. 8 and can operate in a generally similar or identical manner to the system 800.

In the illustrated embodiment, the system 900 includes a reactor vessel 920 and a containment vessel 910 surrounding/enclosing the reactor vessel 920. In some embodiments, the reactor vessel 920 and the containment vessel 910 can be roughly cylinder-shaped or capsule-shaped. The system 900 further includes a plurality of heat pipe layers 911 within the reactor vessel 920. In the illustrated embodiment, the heat pipe layers 911 are spaced apart from and stacked over one another. In some embodiments, the heat pipe layers 911 can be mounted/secured to a common frame 912, a portion of the reactor vessel 920 (e.g., a wall thereof), and/or other suitable structures within the reactor vessel 920. In other embodiments, the heat pipe layers 911 can be directly stacked on top of one another such that each of the heat pipe layers 911 supports and/or is supported by one or more of the other ones of the heat pipe layers 911.

In the illustrated embodiment, the system 900 further includes a shield or reflector region 914 at least partially surrounding a core region 916. The heat pipes layers 911 can be circular, rectilinear, polygonal, and/or can have other shapes, such that the core region 916 has a corresponding three-dimensional shape (e.g., cylindrical, spherical). In some embodiments, the core region 916 is separated from the reflector region 914 by a core barrier 915, such as a metal wall. The core region 916 can include one or more fuel sources, such as fissile material, for heating the heat pipes layers 911. The reflector region 914 can include one or more materials configured to contain/reflect products generated by burning the fuel in the core region 916 during operation of the system 900. For example, the reflector region 914 can include a liquid or solid material configured to reflect neutrons and/or other fission products radially inward toward the core region 916. In some embodiments, the reflector region 914 can entirely surround the core region 916. In other embodiments, the reflector region 914 may partially surround the core region 916. In some embodiments, the core region 916 can include a control material 917, such as a moderator and/or coolant. The control material 917 can at least partially surround the heat pipe layers 911 in the core region 916 and can transfer heat therebetween.

In the illustrated embodiment, the system 900 further includes at least one heat exchanger 930 (e.g., a steam generator) positioned around the heat pipe layers 911. The heat pipe layers 911 can extend from the core region 916 and at least partially into the reflector region 914 and are thermally coupled to the heat exchanger 930. In some embodiments, the heat exchanger 930 can be positioned outside of or partially within the reflector region 914. The heat pipe layers 911 provide a heat transfer path from the core region 916 to the heat exchanger 930. For example, the heat pipe layers 911 can each include an array of heat pipes that provide a heat transfer path from the core region 916 to the heat exchanger 930. When the system 900 operates, the fuel in the core region 916 can heat and vaporize a fluid within the heat pipes in the heat pipe layers 911, and the fluid can carry the heat to the heat exchanger 930. The heat pipes in the heat pipe layers 911 can then return the fluid toward the core region 916 via wicking, gravity, and/or other means to be heated and vaporized once again.

In some embodiments, the heat exchanger 930 can be similar to the steam generator 830 of FIG. 8 and, for example, can include one or more helically-coiled tubes that wrap around the heat pipe layers 911. The tubes of the heat exchanger 930 can include or carry a working fluid (e.g., a coolant such as water or another fluid) that carries the heat from the heat pipe layers 911 out of the reactor vessel 920 and the containment vessel 910 for use in generating electricity, steam, and/or the like. For example, in the illustrated embodiment the heat exchanger 930 is operably coupled to a turbine 943, a generator 944, a condenser 945, and a pump 946. As the working fluid within the heat exchanger 930 increases in temperature, the working fluid may begin to boil and vaporize. The working fluid (e.g., steam) may be used to drive the turbine 943 to convert the thermal potential energy of the working fluid into electrical energy via the generator 944. The condenser 945 can condense the working fluid after it passes through the turbine 943, and the pump 946 can direct the working fluid back to the heat exchanger 930 where it can begin another thermal cycle. In other embodiments, steam from the heat exchanger 930 can be routed for direct use in an industrial process, such as a resource production plant, described in detail above. Accordingly, steam exiting the heat exchanger 930 can bypass the turbine 943, the generator 944, the condenser 945, the pump 946, etc.

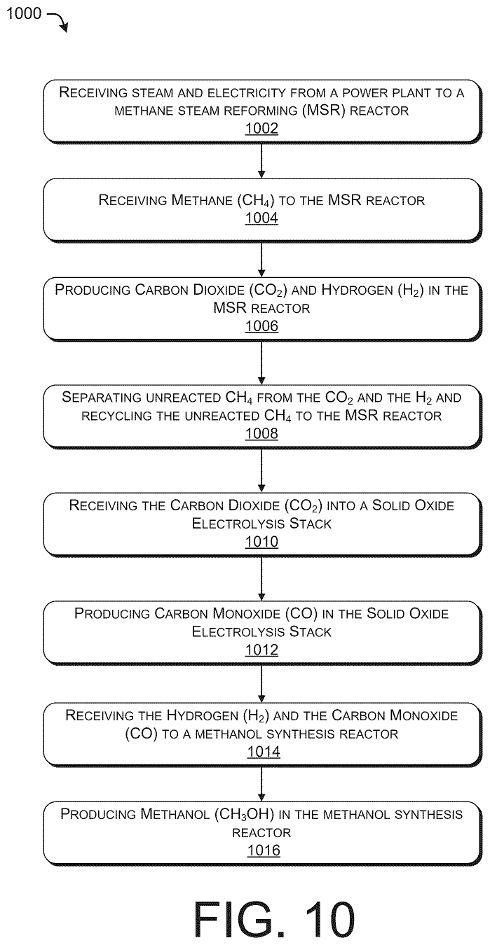

FIG. 10 illustrates an example Low-Temperature Methane steam reforming process 1000 to produce hydrogen and carbon dioxide for methanol production, in accordance with embodiments of the present disclosure. In various examples, the process 1000 can be performed by an Integrated Energy System (IES). The IES may include a power plant, such as the power plant 202 and the power plant system 750. The IES may also include a resource production plant, such as the Low-Temperature MSR Plant 204, the Electrolysis plant 208, and the Methanol synthesis reactor 212. In embodiments, the Low-Temperature MSR Plant 204, the Electrolysis plant 208, and the Methanol synthesis reactor 212 are configured to receive electricity from the power plant.

At 1002, electricity and steam are received from a power plant, such as the power plant 202 and the power plant system 750, to a Low-Temperature MSR reactor. In embodiments, the power plant produces electricity and steam from nuclear energy. For example, the power plant may include one or more small modular nuclear reactor (SMRs) as described elsewhere in this disclosure. In embodiments, the power plant may be local to the resource production plant. For example, the power plant may be located within a threshold distance of 0.4 km (0.25 mile), within 0.8 km (0.5 mile), within 3.22 km (2 miles), within 4.82 km (3 miles), or within 8.1 km (5 miles) of the resource production plant it supports. In embodiments, the power plant is configured to supply a portion of electricity to a power grid.

At 1004, Methane (CH₄) is received to a Low-Temperature MSR reactor. In embodiments, the Low-Temperature MSR reactor may include the Low-Temperature MSR reactor 404 as described in FIG. 4. At 1006, Carbon Dioxide (CO₂) and Hydrogen (H₂) are produced in the Low-Temperature MSR reactor in accordance with methods described herein. For example, the reaction conditions within the production of Carbon Dioxide (CO₂) and Hydrogen (H₂) follows Equation 1 and occurs at an operating temperature in the range of about 200-500° C., a pressure of about 1-50 bar.

At 1008, the unreacted Methane (CH₄) is separated from the Carbon Dioxide (CO₂) and the Hydrogen (H₂), and the unreacted Methane (CH₄) is recycled back to the MSR reactor.

At 1010, the Carbon Dioxide (CO₂) produced in 1002 is received into a Solid Oxide Electrolysis Stack, such as the Solid Oxide Electrolysis Stack 502 described in FIG. 5. At 1012, Carbon Monoxide (CO) is produced in the Solid Oxide Electrolysis Stack in accordance with methods described herein.

At 1014, the Hydrogen (H₂) from 1006 and the Carbon Monoxide (CO) from 1012 are received in a methanol synthesis reactor, such as the Methanol Synthesis Reactor 602 described in FIG. 6. At 1016, Methanol (CH₃OH) is produced in the methanol synthesis reactor in accordance with methods described herein.

### CONCLUSION

While the foregoing invention is described with respect to the specific examples, it is to be understood that the scope of the invention is not limited to these specific examples. Since other modifications and changes varied to fit particular operating requirements and environments will be apparent to those skilled in the art, the invention is not considered limited to the example chosen for purposes of disclosure and covers all changes and modifications which do not constitute departures from the true spirit and scope of this invention.

Although the application describes embodiments having specific structural features and/or methodological acts, it is to be understood that the claims are not necessarily limited to the specific features or acts described. Rather, the specific features and acts are merely illustrative some embodiments that fall within the scope of the claims.

What is claimed is:

1. An Integrated Energy System (IES) comprising:
a power plant configured to generate steam;
a methane steam reforming (MSR) plant configured to receive Methane (CH₄) and at least a first portion of the steam, and to produce Carbon Dioxide (CO₂), Hydrogen (H₂), and first Carbon Monoxide (CO);
a gas separation plant configured to separate the Carbon Dioxide (CO₂), the first Carbon Monoxide (CO), and Hydrogen (H₂) from unreacted steam and unreacted Methane (CH₄);
an electrolysis plant configured to receive the Carbon Dioxide (CO₂) and to produce second Carbon Monoxide (CO) and Oxygen (O₂); and
a methanol synthesis reactor configured to receive the Hydrogen (H₂), the first Carbon Monoxide (CO), and the second Carbon Monoxide (CO) to produce Methanol (CH₃OH).

2. The IES of claim 1, wherein the power plant comprises at least one nuclear reactor and electrical power generation system, the at least one nuclear reactor being configured to generate the steam, and the electrical power generation system being configured to generate electricity.

3. The IES of claim 2, wherein the MSR plant receives at least a portion of the electricity from the power plant.

4. The IES of claim 1, wherein the MSR plant comprises:
a reactor configured to receive the Methane (CH₄) and water (H₂O) to produce the Carbon Dioxide (CO₂), the Hydrogen (H₂), and the first Carbon Monoxide (CO),
wherein the first portion of the steam from the power plant provides thermal energy to the reactor.

5. The IES of claim 4, wherein the water (H₂O) comprises the first portion of the steam from the power plant.

6. The IES of claim 4, wherein the MSR plant further comprises a first separation unit configured to separate the Carbon Dioxide (CO₂), the Hydrogen (H₂), and the first Carbon Monoxide (CO).

7. The IES of claim 1, wherein the electrolysis plant comprises a Solid Oxide Electrolysis Stack configured to produce the second Carbon Monoxide (CO) and the Oxygen (O₂).

8. The IES of claim 7, wherein the electrolysis plant further comprises:
a first separation unit configured to separate the Carbon Dioxide (CO₂) from the second Carbon Monoxide (CO) produced in the Solid Oxide Electrolysis Stack; and
a second separation unit configured to separate the Carbon Dioxide (CO₂) from the Oxygen (O₂) produced in the Solid Oxide Electrolysis Stack.

9. The IES of claim 2, wherein the electrolysis plant is configured to receive at least a portion of the electricity from the power plant.

10. The IES of claim 2, wherein the methanol synthesis reactor is configured to receive at least a portion of the electricity from the power plant.

11. An Integrated Energy System (IES) comprising:
a power plant configured to generate steam;
a methane steam reforming (MSR) reactor configured to receive at least a portion of the steam (H₂O) from the power plant to react with Methane (CH₄) within the MSR reactor to produce Hydrogen (H₂) and Carbon Dioxide (CO₂);
a first separation unit configured to separate unreacted steam from the Hydrogen (H₂) and the Carbon Dioxide (CO₂);
a second separation unit configured to separate the Hydrogen (H₂) from the Carbon Dioxide (CO₂);
a third separation unit configured to separate unreacted Methane (CH₄) from the Carbon Dioxide (CO₂);
a Solid Oxide Electrolysis Stack configured to receive at least a portion of the Carbon Dioxide (CO₂) and to produce Carbon Monoxide (CO);
a second separation unit configured to separate the Carbon Dioxide (CO₂) from the Carbon Monoxide (CO); and
a methanol synthesis reactor configured to receive at least a portion of the Hydrogen (H₂) and at least a portion of the Carbon Monoxide (CO) to produce Methanol (CH₃OH).

12. The IES of claim 11, wherein the power plant comprises at least one nuclear reactor and electrical power generation system, the at least one nuclear reactor being configured to generate the steam, and the electrical power generation system being configured to generate electricity.

13. The IES of claim 11, wherein the MSR reactor is configured to receive unreacted Methane (CH₄) separated in the third separation unit.

14. The IES of claim 12, wherein the MSR reactor is configured to receive at least a first portion of the electricity from the power plant and the methanol synthesis reactor is configured to receive at least a second portion of the electricity from the power plant.

15. A method comprising:
receiving steam and electricity from a power plant to a methane steam reforming (MSR) reactor;
receiving Methane (CH₄) to the MSR reactor to react with at least a portion of the steam to produce Carbon Dioxide (CO₂) and Hydrogen (H₂);
receiving the Carbon Dioxide (CO₂), the Hydrogen (H₂), unreacted Methane (CH₄), and unreacted steam to at least one separator;
receiving the Carbon Dioxide (CO₂) into a Solid Oxide Electrolysis Stack to produce Carbon Monoxide (CO); and
receiving the Hydrogen (H₂) and the Carbon Monoxide (CO) to a methanol synthesis reactor configured to produce Methanol (CH₃OH).

16. The method of claim 15, wherein the power plant comprises at least one nuclear reactor and electrical power generation system, the at least one nuclear reactor being configured to generate steam, and the electrical power generation system being configured to generate the electricity.

17. The method of claim 15, further comprising maintaining the MSR reactor at a temperature in a range of 200-500° C. and a pressure in a range of 1-50 bar.

18. The method of claim 15, wherein the unreacted steam is separated in a first separation stage, the Hydrogen (H₂) is separated in a second separation stage, and the Methane (CH₄) and Carbon Dioxide (CO2) are separated in a third separation stage.

19. The method of claim 18, wherein the Methane (CH₄) is recycled to the MSR reactor.

20. The method of claim 15, further comprising maintaining the methanol synthesis reactor at a temperature between 20° and 300° C. and a pressure between 50 and 100 bar.
